(19) United States
(12) Patent Application Publication
(10) Pub. No.: US 2001/0018041 A1
(43) Pub. Date: Aug. 30, 2001

(54) Treatment of B cell malignancies using anti-CD40L antibodies in combination with anti-CD20 antibodies and/or chemotherapeutics and radiotherapy

(75) Inventors: Nabil Hanna, Rancho Santa Fe, CA (US); Kandasamy Hariharan, San Diego, CA (US)

Correspondence Address: Intellectual Property Group, Pillsbury Winthrop LLP, East Tower, Ninth Floor, 1100 New York Avenue, N.W., Washington, DC, 20005-3918, US

(73) Assignee: IDEC Pharmaceuticals Corporation

(21) Appl. No.: 09/834,933

(22) Filed: Apr. 16, 2001

Related U.S. Application Data
(62) Division of application No. 09/435,992, filed on Nov. 8, 1999.

(51) Int. Cl. A61K 39/395

(52) U.S. Cl. 424/1.49; 424/181.1

(57) ABSTRACT

The invention discloses compositions, combination therapies and methods of treating B-cell lymphomas and leukemias, as well as other CD40+ malignancies. The primary active agent of the composition is an anti-CD40L antibody or other CD40L antagonist that inhibits CD40-CD40L interaction. Compositions may additionally contain or utilize any one or more of the following in combination for the treatment of said disease: anti-CD20 antibodies, chemotherapeutic agents, chemotherapy cocktails, and radiotherapy.

### FIELD OF THE INVENTION

The invention describes a method and combination therapy for treating B-cell lymphomas and leukemias, as well as other CD40+ malignancies, by regulating the interaction between CD40 and its ligand, CD40L or regulating CD40 signaling. Specifically, the interaction can be inhibited using anti-CD40L antibodies to prevent CD40L from binding to CD40. These antibodies or other agents which can inhibit CD40/CD40L interaction further can be combined with chemotherapeutics, radiation and/or anti-CD20 antibodies and anti-CD40 antibodies.

### BACKGROUND OF THE INVENTION

Lymphomas are tumors of the lymphocytes. Ninety percent of lymphomas are of B-cell origin, with the remaining ten percent of T-cell origin. Most patients are diagnosed with either Hodgkin's Disease (HD) or non-Hodgkin's type lymphoma (NHL).

Depending on the lymphoma diagnosed, treatment options include radiotherapy, chemotherapy, and use of monoclonal antibodies.

A. Anti-CD20 Antibodies

CD20 is a cell surface antigen expressed on more than 90% of B-cell lymphomas, which does not shed or modulate in the neoplastic cells (McLaughlin et al., J. Clin. Oncol. 16: 2825-2833 (1998b)). The CD20 antigen is a non-glycosylated, 35 kDa B-cell membrane protein involved in intracellular signaling, B-cell differentiation and calcium channel mobilization (Clark et al., Adv. Cancer Res. 52: 81-149 (1989); Tedder et al., Immunology Today 15: 450-454 (1994)). The antigen appears as an early marker of the human B-cell lineage, and is ubiquitously expressed at various antigen densities on both normal and malignant B-cell populations. However, the antigen is absent on fully, mature B-cells (e.g., plasma cells), early B-cell populations and stem cells, making it a suitable target for antibody mediated therapy.

Anti-CD20 antibodies have been prepared for use both in research and therapeutics. One anti-CD20 antibody is the monoclonal B1 antibody (U.S. Pat. No. 5,843,398). Anti-CD20 antibodies have also been prepared in the form of radionuclides for treating B-cell lymphoma (e.g., ¹³¹I-labeled anti-CD20 antibody), as well as a ⁸⁹Sr-labeled form for the palliation of bone pain caused by prostate and breast cancer metastasises (Endo, Gan To Kagaku Ryoho 26: 744-748 (1999)).

A murine monoclonal antibody, 1F5, (an anti-CD20 antibody) was reportedly administered by continuous intravenous infusion to B-cell lymphoma patients. However, extremely high levels (>2 grams) of IF5 were reportedly required to deplete circulating tumor cells, and the results were described as “transient” (Press et al., Blood 69: 584-591 (1987)). A potential problem with using monoclonal antibodies in therapeutics is non-human monoclonal antibodies (e.g., murine monoclonal antibodies) typically lack human effector functionality, e.g., they are unable to, inter alia, mediate complement dependent lysis or lyse human target cells through antibody-dependent cellular toxicity or Fc-receptor mediated phagocytosis. Furthermore, non-human monoclonal antibodies can be recognized by the human host as a foreign protein; therefore, repeated injections of such foreign antibodies can lead to the induction of immune responses leading to harmful hypersensitivity reactions. For murine-based monoclonal antibodies, this is often referred to as a Human Anti-Mouse Antibody response, or “HAMA” response. Additionally, these “foreign” antibodies can be attacked by the immune system of the host such that they are, in effect, neutralized before they reach their target site.

RITUXAN®. RITUXAN® (also known as Rituximab, MabThera®, IDEC-C2B8 and C2B8) was the first FDA-approved monoclonal antibody and was developed at IDEC Pharmaceuticals (see U.S. Pat. Nos. 5,843,439; 5,776,456 and 5,736,137) for treatment of human B-cell lymphoma (Reff et al., Blood 83: 435-445 (1994)). RITUXAN® is a chimeric, anti-CD20 monoclonal (MAb) which is growth inhibitory and reportedly sensitizes certain lymphoma cell lines for apoptosis by chemotherapeutic agents in vitro (Demidem et al., Cancer Biotherapy & Radiopharmaceuticals 12: 177-(1997)). RITUXAN® also demonstrates anti-tumor activity when tested in vivo using murine xenograft animal models. RITUXAN® efficiently binds human complement, has strong FcR binding, and can efficiently kill human lymphocytes in vitro via both complement dependent (CDC) and antibody-dependent (ADCC) mechanisms (Reff et al., Blood 83: 435-445 (1994)). In macaques, the antibody selectively depletes normal B-cells from blood and lymph nodes.

RITUXAN® has been recommended for treatment of patients with low-grade or follicular B-cell non-Hodgkin's lymphoma (McLaughlin et al., Oncology (Huntingt) 12: 1763-1777 (1998a); Maloney et al., Oncology 12: 63-76 (1998); Leget et al., Curr. Opin. Oncol. 10: 548-551 (1998)). In Europe, RITUXAN® has been approved for therapy of relapsed stage III/IV follicular lymphoma (White et al., Pharm. Sci. Technol. Today 2:95-101 (1999)) and is reportedly effective against follicular center cell lymphoma (FCC) (Nguyen et al., Eur. J Haematol 62: 76-82 (1999)). Other disorders treated with RITUXAN® include follicular center cell lymphoma (FCC), mantle cell lymphoma (MCL), diffuse large cell lymphoma (DLCL), and small lymphocytic lymphoma/chronic lymphocytic leukemia (SLL/CLL) (Nguyen et al., 1999)). Patients with refractory or incurable NHL reportedly have responded to a combination of RITUXAN® and CHOP (e.g., cyclophosphamide, vincristine, prednisone and doxorubicin) therapies (Ohnishi et al., Gan To Kagaku Ryoho 25: 2223-8 (1998)). RITUXAN® has exhibited minimal toxicity and significant therapeutic activity in low-grade non-Hodgkin's lymphomas (NHL) in phase I and II clinical studies (Berinstein et al., Ann. Oncol. 9: 995-1001 (1998)).

RITUXAN®, which was used alone to treat B-cell NHL at weekly doses of typically 375 mg/M² for four weeks with relapsed or refractory low-grade or follicular NHL, was well tolerated and had significant clinical activity (Piro et al., Ann. Oncol. 10: 655-61 (1999); Nguyen et al., (1999); and Coiffier et al., Blood 92: 1927-1932 (1998)). However, up to 500 mg/M² of four weekly doses have also been administered during trials using the antibody (Maloney et al., Blood 90: 2188-2195 (1997)). RITUXAN® also has been combined with chemotherapeutics, such as CHOP (e.g., cyclophosphamide, doxorubicin, vincristine and prednisone), to treat patients with low-grade or follicular B-cell non-Hodgkin's lymphoma (Czuczman et al., J. Clin. Oncol. 17: 268-76 (1999); and McLaughlin et al., (1998a)).

B. CD40 and CD40L

CD40 is expressed on the cell surface of mature B-cells, as well as on leukemic and lymphocytic B-cells, and on Hodgkin's and Reed-Sternberg (RS) cells of Hodgkin's Disease (HD) (Valle et al., Eur. J. Immunol. 19: 1463-1467 (1989); and Gruss et al., Leuk. Lymphoma 24: 393-422 (1997)). CD40 is a B-cell receptor leading to activation and survival of normal and malignant B-cells, such as non-Hodgkin's follicular lymphoma (Johnson et al., Blood 82: 1848-1857 (1993); and Metkar et al., Cancer Immunol. Immunother. 47: 104 (1998)). Signaling through the CD40 receptor protects immature B-cells and B-cell lymphomas from IgM- or Fas-induced apoptosis (Wang et al., J. Immunology 155: 3722-3725 (1995)). Similarly, mantel cell lymphoma cells have a high level of CD40, and the addition of exogenous CD40L enhanced their survival and rescued them from fludarabin-induced apoptosis (Clodi et al., Brit. J. Haematol. 103: 217-219 (1998)). In contrast, others have reported that CD40 stimulation may inhibit neoplastic B-cell growth both in vitro (Funakoshi et al., Blood 83: 2787-2794 (1994)) and in vivo (Murphy et al., Blood 86: 1946-1953 (1995)).

Anti-CD40 antibodies (see U.S. Pat. Nos. 5,874,082 and 5,667,165) administered to mice increased the survival of mice with human B-cell lymphomas (Funakoshi et al., (1994); and Tutt et al., J. Immunol. 161: 3176-3185 (1998)). Methods of treating neoplasms, including B-cell lymphomas and EBV-induced lymphomas using anti-CD40 antibodies mimicking the effect of CD40L and thereby delivering a death signal, are described in U.S. Pat. No. 5,674,492 (1997), which is herein incorporated by reference in its entirety. CD40 signaling has also been associated with a synergistic interaction with CD20 (Ledbetter et al., Circ. Shock 44: 67-72 (1994)). Additional references describing preparation and use of anti-CD40 antibodies include U.S. Pat. Nos. 5,874,085 (1999), 5,874,082 (1999), 5,801,227 (1998), 5,674,492 (1997) and 5,667,165 (1997), which are incorporated herein by reference in their entirety.

A CD40 ligand, gp39 (also called CD40 ligand, CD40L or CD154), is expressed on activated, but not resting, CD4+ Th cells (Spriggs et al., J. Exp. Med. 176: 1543-1550 (1992); Lane et al., Eur. J. Immunol. 22: 2573-2578 (1992); and Roy et al., J. Immunol. 151: 1-14 (1993)). Both CD40 and CD40L have been cloned and characterized (Stamenkovi et al., EMBO J. 8: 1403-1410 (1989); Armitage et al., Nature 357: 80-82 (1992); Lederman et al., J. Exp. Med. 175: 1091-1101 (1992); and Hollenbaugh et al., EMBO J. 11: 4313-4321 (1992)). Human CD40L is also described in U.S. Pat. No. 5,945,513. Cells transfected with the CD40L gene and expressing the CD40L protein on their surface can trigger B-cell proliferation, and together with other stimulatory signals, can induce antibody production (Armitage et al., (1992); and U.S. Pat. No. 5,945,513). CD40L may play an important role in the cell contact-dependent interaction of tumor B-cells (CD40+) within the neoplastic follicles or Reed-Sternberg cells (CD40+) in Hodgkin's Disease areas (Carbone et al., Am. J. Pathol. 147: 912-922 (1995)). Anti-CD40L monoclonal antibodies reportedly have been effectively used to inhibit the induction of murine AIDS (MAIDS) in LP-BM5-infected mice (Green et al., Virology 241: 260-268 (1998)). However, the mechanism of CD40L-CD40 signaling leading to survival versus cell death responses of malignant B-cells is unclear. For example, in follicular lymphoma cells, down-regulation of a apoptosis inducing TRAIL molecule (APO-2L) (Ribeiro et al., British J. Haematol 103: 684-689 (1998)) and over expression of BCL-2, and in the case of B-CLL, down-regulation of CD95 (Fas/APO-1) (Laytragoon-Lewin et al., Eur. J Haematol. 61: 266-271 (1998)) have been proposed as mechanisms of survival. In contrast, evidence exists in follicular lymphoma, that CD40 activation leads to up-regulation of TNF (Worm et al., International Immunol. 6: 1883-1890 (1994)) CD95 molecules (Plumas et al., Blood 91: 2875-2885 (1998)).

Anti-CD40 antibodies have also been prepared to prevent or treat antibody-mediated diseases, such as allergies and autoimmune disorders as described in U.S. Pat. No.5,874,082 (1999). Anti-CD40 antibodies reportedly have been effectively combined with anti-CD20 antibodies yielding an additive effect in inhibiting growth of non-Hodgkin's B-cell lymphomas in cell culture (Benoit et al., Immunopharmacology 35: 129-139 (1996)). In vivo studies in mice purportedly demonstrated that anti-CD20 antibodies were more efficacious than anti-CD40 antibodies administered individually in promoting the survival of mice bearing some, but not all, lymphoma lines (Funakoshi et al., J. Immunother. Emphasis Tumor Immunol. 19: 93-101 (1996)). Anti-CDl9 antibodies are reportedly also effective in vivo in the treatment of two syngeneic mouse B-cell lymphomas, BCL1 and A31 (Tutt et al. (1998)). Antibodies to CD40L have also been described for use to treat disorders associated with B-cell activation (European Patent No. 555,880 (1993)). Anti-CD40L antibodies include monoclonal antibodies 3E4, 2H5, 2H8, 4D9-8, 4D9-9, 24-31, 24-43, 89-76 and 89-79, as described in U.S. Pat. No. 5,7474,037 (1998), and anti-CD40L antibodies described in U.S. Pat. No. 5,876,718 (1999) used to treat graft-versus-host-disease.

Therefore, not withstanding what has previously been reported in the literature, there exists a need for improved methods of treating and combination therapies for treating B-cell lymphomas and leukemias. Use of compositions containing anti-CD40L antibodies, and other agents which antagonize CD40-CD40L interactions, offers another avenue of treatment to cancer patients, which may be less toxic than existing therapies. Specifically, the proposed methods and compositions for inhibiting CD40 stimulation to prevent cancer cells from becoming refractory to programmed cell death caused by chemotherapy or other cancer therapies, offers a previously unknown method of enhancing cancer therapy and reducing the potential of developing cells resistant to therapy.

### SUMMARY OF THE INVENTION

It is an object of the invention to provide a method for treating B-cell lymphomas, B-cell leukemias, and other CD40+ malignancies comprising administering a therapeutically effective amount of an antibody or antibody fragment which binds to CD40L. The B-cell lymphomas include Hodgkin's lymphoma and non-Hodgkin's lymphomas of any grade.

Another object of the invention is to provide a combination therapy for the treatment of a B-cell lymphoma or a B-cell leukemia comprising an anti-CD40L antibody or antibody fragment or CD40L antagonist and at least one of the following (a) a chemotherapeutic agent or a combination of chemotherapeutic agents, (b) radiotherapy, (c) an anti-CD20 antibody or fragment thereof and (d) an anti-CD40 antibody or fragment thereof.

### BRIEF DESCRIPTION OF THE DRAWINGS

FIG. 1. Sensitivity of B-lymphoma cells to adriamycin after 4 hour exposure.

FIG. 2. (Panel A) Anti-CD40L (IDEC- 13 1) overrides CD40L mediated resistance to killing by ADM of B-lymphoma cells. (Panel B) Effect of RITUXAN® on normal and sCD40L pre-treated DHL-4 cells.

FIG. 3. (Panel A) Blocking of CD40L mediated cell survival of B-CLL by anti-CD40L antibody (IDEC-131). (Panel B) Blocking of CD40L mediated survival of B-CLL by IDEC's C2B8.

FIG. 4. FACS analysis comparing HLA-DR expression in CD19+ CLL cells cultured with sCD40L and cells not cultured with sCD40L.

### DETAILED DESCRIPTION OF THE INVENTION

Another aspect of the invention is to provide a composition for treating leukemias and lymphomas, as well as other malignancies which express CD40. A preferred embodiment of the invention, are compositions and methods of their use to treat lymphomas and leukemias of the B-cell lineage. The compositions may comprise agents, which antagonize CD40 signaling or the interaction between CD40 and CD40L. The agents optionally may comprise one active agent, such as an anti-CD40L antibody or fragment thereof, as well as peptide fragments, peptide mimetics, or chemical compounds. Alternatively, the composition may comprise multiple active agents, which target aspects of the malignancy other than CD40 signaling or CD40/CD40L interaction, such as chemotherapeutics, other antibodies, and/or may be administered in combination with radiation therapy.

A. Definitions

The term “CD40L antibody” as used herein is intended to include immunoglobulins and fragments thereof which are specifically reactive with a CD40L protein or peptide thereof or a CD40L fusion protein. CD40L antibodies can include human antibodies, primatized antibodies, chimeric antibodies, bispecific antibodies and humanized antibodies.

By “CD40 antibody” as used herein is intended to include immunoglobulins and fragments thereof which are specifically reactive with a CD40 protein or peptide thereof or a CD40 fusion protein. CD40 antibodies can include human antibodies, primatized antibodies, chimeric antibodies, bispecific antibodies and humanized antibodies.

BY “CD20 antibody” as used herein is intended to include immunoglobulins and fragments thereof which are specifically reactive with a CD20 protein or peptide thereof or a CD20 fusion protein. CD20 antibodies can include human antibodies, primatized antibodies, chimeric antibodies, bispecific antibodies and humanized antibodies. Anti-CD20 antibodies include the monoclonal B I antibody and RITUXAN®.

By “humanized antibody” is meant an antibody derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting only the non-human complementarity determining regions (CDRs) into human framework and constant regions with or without retention of critical framework residues; and (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); and Padlan, Molec. Immun. 31: 169-217 (1994), all of which are hereby incorporated by reference in their entirety. Humanized anti-CD40L antibodies can be prepared as described in U.S. patent application Ser. No. 08/554,840 filed Nov. 7, 1995 also incorporated herein by reference in its entirety.

By “human antibody” is meant an antibody containing entirely human light and heavy chain as well as constant regions, produced by any of the known standard methods.

By “primatized antibody” is meant a recombinant antibody which has been engineered to contain the variable heavy and light domains of a monkey (or other primate) antibody, in particular, a cynomolgus monkey antibody, and which contains human constant domain sequences, preferably the human immunoglobulin gamma 1 or gamma 4 constant domain (or PE variant). The preparation of such antibodies is described in Newman et al., Biotechnology, 10: 1458-1460 (1992); also in commonly assigned 08/379,072, 08/487,550, or 08/746,361, all of which are incorporated by reference in their entirety herein. These antibodies have been reported to exhibit a high degree of homology to human antibodies, i.e., 85-98%, display human effector functions, have reduced immunogenicity, and may exhibit high affinity to human antigens.

By “antibody fragment” is meant an fragment of an antibody such as Fab, F(ab′)₂, Fab′ and scFv.

By “chimeric antibody” is meant an antibody containing sequences derived from two different antibodies, which typically are of different species. Most typically, chimeric antibodies comprise human and murine antibody fragments, and generally human constant and murine variable regions.

By “bispecific antibody” is meant an antibody molecule with one antigen-binding site specific for one antigen, and the other antigen-binding site specific for another antigen.

By “immunogenicity” is meant the ability of a targeting protein or therapeutic moiety to elicit an immune response (e.g., humoral or cellular) when administered to a subject.

It is especially advantageous to formulate parenteral compositions in dosage unit form for ease of administration and uniformity of dosage. “Dosage unit form,” as used herein, refers to physically discrete units suited as unitary dosages for the mammalian subjects to be treated; each unit containing a predetermined quantity of active compound is calculated to produce the desired therapeutic effect in association with the required pharmaceutical carrier. The specification for the dosage unit forms of the invention are dictated by and directly dependent on: (A) the unique characteristics of the active compound and the particular therapeutic effect to be achieved; and (B) the limitations inherent in the art of compounding such an active compound for the treatment of sensitivity in individuals.

B. CD40L Antagonists

According to the methods of the invention, a CD40L antagonist is administered to a subject to interfere with the interaction of CD40L and its binding partner, CD40. A “CD40L antagonist” is defined as a molecule which interferes with this interaction. The CD40L antagonist can be an antibody directed against CD40L (e.g., a monoclonal antibody against CD40L), a fragment or derivative of an antibody against CD40L (e.g., Fab or F(ab)′₂ fragments), chimeric antibodies or humanized antibodies, soluble forms of CD40, soluble forms of a fusion protein comprising CD40, or pharmaceutical agents which disrupt or interfere with the CD40L-CD40 interaction or interferes with- CD40 signaling.

Antibodies. To prepare anti-CD40L antibodies, a mammal (e.g., a mouse, hamster, rabbit or ungulate) can be immunized with an immunogenic form of CD40L protein or protein fragment (e.g., peptide fragment), which elicits an antibody response in the animal. A cell expressing CD40L on its surface can also be utilized as an immunogen. Alternative immunogens include purified CD40L protein or protein fragments. CD40L can be purified from a CD40L-expressing cell by standard purification techniques (Armitage et al., (1992); Lederman et al., (1992); and Hollenbaugh et al., (1992)). Alternatively, CD40L peptides can be prepared based upon the amino acid sequence of CD40L, as disclosed in Armitage et al., (1992). Techniques for conferring immunogenicity on a protein include conjugation to carriers or other techniques well known in the art. For example, the protein can be administered in the presence of an adjuvant. The process of immunization can be monitored by detection of antibody titers in plasma or serum. Standard ELISA or other immunoassays can be used with the immunogen as antigen to assess the levels of antibodies. Following immunization, antisera can be obtained and polyclonal antibodies isolated. To produce monoclonal antibodies, antibody producing cells can be harvested and fused with myeloma cells using standard somatic cell fusion procedures, as described in U.S. Pat. Nos. 5,833,987 (1998) and 5,747,037 (1997).

Antibodies can be fragments using conventional techniques, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. Other antibody fragments contemplated include Fab and scFv.

One method of minimizing recognition of non-human antibodies when used therapeutically in humans, other than general immunosuppression, is to produce chimeric antibody derivatives, i.e., antibody molecules that combine a non-human animal variable region and a human constant region. The humanized chimeric antibody molecules can include, for example, the antigen binding domain from an antibody of a mouse, rat or other species, with human constant regions. Methods for making these humanized chimeric antibodies include those references cited in U.S. Pat. No. 5,833,987 (1998).

For human therapeutic purposes, the antibodies specifically reactive with a CD40L protein or peptide can be further humanized by producing human variable region chimeras, in which parts of the variable regions, especially the conserved framework regions of the antigen-binding domain, are of human origin and only the hypervariable regions are of non-human origin. Such altered immunoglobulin molecules may be made by any of several techniques known in the art, (e.g., Teng et al., Proc. Nati. Acad. Sci. U.S.A. 80: 7308-7312 (1983); Kozbor et al., Immunology Today 4: 7279 (1983); Olsson et al., Meth. Enzymol. 92: 3-16 (1982)), and are preferably made according to the teachings of PCT Publication WO 92/06193 or EP 0239400. Humanized antibodies can be commercially produced by, for example, Scotgen Limited, 2 Holly Road, Twickenham, Middlesex, Great Britain. A preferred humanized gp39 (CD40L) antibody, IDEC-131, is disclosed in allowed U.S. application Ser. No. 08/554,840, incorporated by reference in its entirety herein.

Another method of generating specific antibodies, or antibody fragments, reactive against a CD40L protein or peptide (e.g., such as the gp39 fusion protein described in U.S. Pat. No. 5,945,513) is to screen expression libraries encoding immunoglobulin genes, or portions thereof, expressed in bacteria with a CD40L protein or peptide. For example, complete Fab fragments, V_{H} regions and Fv regions can be expressed in bacteria using phage expression libraries. See for example, Ward et al., Nature 341: 544-546 (1989); Huse et al., Science 246: 1275-1281 (1989); and McCafferty et al., Nature 348: 552-554 (1990). Screening such libraries with, for example, a CD40L peptide, can identify immunoglobulin fragments reactive with CD40L. Alternatively, the SCID-hu mouse (available from Genpharm) can be used to produce antibodies or fragments thereof.

Methodologies for producing monoclonal antibodies (MAb) directed against CD40L, including human CD40L and mouse CD40L, and suitable monoclonal antibodies for use in the methods of the invention, are described in PCT patent application Ser. No. WO 95/06666 entitled “Anti-gp39 Antibodies and Uses Therefor;” the teachings of which are incorporated herein by reference in their entirety. Particularly preferred anti-human CD40L antibodies of the invention are MAbs 24-31 and 89-76, produced respectively by hybridomas 24-31 and 89-76. The 89-76 and 24-31 hybridomas, producing the 89-76 and 24-31 antibodies, respectively, were deposited under the provisions of the Budapest Treaty with the American Type Culture Collection (ATCC), 10801 University Blvd., Manassas, Va. 20110-2209, on Sep. 2, 1994. The 89-76 hybridoma was assigned ATCC Accession Number HB11713 and the 24-31 hybridoma was assigned ATCC Accession Number HB11712.

Recombinant anti-CD40L antibodies, such as chimeric and humanized antibodies, can be produced by manipulating a nucleic acid (e.g., DNA or cDNA) encoding an anti-CD40L antibody according to standard recombinant DNA techniques. Accordingly, another aspect of this invention pertains to isolated nucleic acid molecules encoding immunoglobulin heavy or light chains, or portions thereof, reactive with CD40L, particularly human CD40L. The immunoglobulin-encoding nucleic acid can encode an immunoglobulin light (V_{L}) or heavy (V_{H}) chain variable region, with or without a linked heavy or light chain constant region (or portion thereof). Such nucleic acids can be isolated from a cell (e.g., hybridoma) producing an anti-human CD40L MAb by standard techniques. For example, nucleic acids encoding the 24-31 or 89-76 MAb can be isolated from the 24-31 or 89-76 hybridomas, respectively, by cDNA library screening, PCR amplification or other standard techniques. Moreover, nucleic acids encoding an anti-human CD40L MAb can be incorporated into an expression vector and introduced into a suitable host cell to facilitate expression and production of recombinant forms of anti-human CD40L antibodies.

Primatized Antibodies. Another highly efficient means for generating recombinant antibodies is disclosed by Newman, Biotechnology, 10: 1455-1460 (1992). More particularly, this technique results in the generation of primatized antibodies which contain monkey variable domains and human constant sequences. This reference is incorporated by reference in its entirety herein. Moreover, this technique is also described in commonly assigned U.S. application Ser. No. 08/379,072, filed on Jan. 25, 1995, which is a continuation of U.S. application Ser. No. 07/912,292, filed Jul. 10, 1992, which is a continuation-in-part of U.S. application Ser. No. 07/856,281, filed Mar. 23, 1992, which is finally a continuation-in-part of U.S. application Ser. Nos. 07/735,064, filed Jul. 25, 1991. 08/379,072 and the parent application thereof all of which are incorporated by reference in their entirety herein.

This technique modifies antibodies such that they are not antigenically rejected upon administration in humans. This technique relies on immunization of cynomolgus monkeys with human antigens or receptors. This technique was developed to create high affinity monoclonal antibodies directed to human cell surface antigens.

Identification of macaque antibodies to human CD40L by screening of phage display libraries or monkey heterohybridomas obtained using B lymphocytes from CD40L immunized monkeys can be performed using the methods described in commonly assigned U.S. application Ser. No. 08/487,550, filed Jun. 7, 1995, incorporated by reference in its entirety herein.

Antibodies generated using the methods described in these applications have previously been reported to display human effector function, have reduced immunogenicity, and long serum half-life. The technology relies on the fact that despite the fact that cynomolgus monkeys are phylogenetically similar to humans, they still recognize many human proteins as foreign and therefore mount an immune response. Moreover, because the cynomolgus monkeys are phylogenetically close to humans, the antibodies generated in these monkeys have been discovered to have a high degree of amino acid homology to those produced in humans. Indeed, after sequencing macaque immunoglobulin light and heavy chain variable region genes, it was found that the sequence of each gene family was 85-98% homologous to its human counterpart (Newman et al., 1992). The first antibody generated in this way, an anti-CD4 antibody, was 91-92% homologous to the consensus sequence of human immunoglobulin framework regions (Newman et al., 1992).

As described above, the present invention relates, in part, to the identification of monoclonal antibodies or primatized forms thereof which are specific to human CD40L antigen and which are capable of inhibiting CD40 signaling or inhibiting CD40/CD40L interaction. Blocking of the primary activation site between CD40 and CD40L with the identified antibodies (or therapeutically effective fragments thereof), while allowing the combined antagonistic effect on positive co-stimulation with an agnostic effect on negative signaling will be a useful therapeutic approach for intervening in relapsed forms of malignancy, especially B-cell lymphomas and leukemias. The functional activity of the identified antibodies is defined by blocking the signals of CD40 permitting it to survive and avoid IgM- or Fas-induced apoptosis.

Manufacture of novel monkey monoclonal antibodies which specifically bind human CD40L or CD40, as well as primatized antibodies derived therefrom can be performed using the methods described in co-pending U.S. application Ser. No. 08/487,550, and as set forth herein. These antibodies possess high affinity to CD40L and therefore may be used as immunosuppressants which inhibit the CD40L/CD40 pathway.

Preparation of monkey monoclonal antibodies will preferably be effected by screening of phage display libraries or by preparation of monkey heterohybridomas using B lymphocytes obtained from CD40L (e.g., human CD40) immunized monkeys. The human CD40 can also be from the fusion protein described in U.S. Pat. No. 5,945,513.

As noted, the first method for generating anti-CD40L antibodies involves recombinant phage display technology. This technique is generally described supra.

Essentially, this will comprise synthesis of recombinant immunoglobulin libraries against CD40L antigen displayed on the surface of filamentous phage and selection of phage which secrete antibodies having high affinity to CD40L antigen. As noted supra, preferably antibodies will be selected which bind to both human CD40L and CD40. To effect such methodology, the present inventors have created a unique library for monkey libraries which reduces the possibility of recombination and improves stability.

Essentially, to adopt phage display for use with macaque libraries, this vector contains specific primers for PCR amplifying monkey immunoglobulin genes. These primers are based on macaque sequences obtained while developing the primatized technology and databases containing human sequences.

Suitable primers are disclosed in commonly assigned U.S. Ser. No. 08/379,072, incorporated by reference herein.

The second method involves the immunization of monkeys, i.e., macaques, against human CD40L antigen. The inherent advantage of macaques for generation of monoclonal antibodies is discussed supra. In particular, such monkeys, i.e., cynomolgus monkeys, may be immunized against human antigens or receptors. Moreover, the resultant antibodies may be used to make primatized antibodies according to the methodology of Newman et al., (1992), and Newman et al., commonly assigned U.S. Ser. No. 08/379,072, filed Jan. 25, 1995, which are incorporated by reference in their entirety.

The significant advantage of antibodies obtained from cynomolgus monkeys is that these monkeys recognize many human proteins as foreign and thereby provide for the formation of antibodies, some with high affinity to desired human antigens, e.g., human surface proteins and cell receptors. Moreover, because they are phylogenetically close to humans, the resultant antibodies exhibit a high degree of amino acid homology to those produced in humans. As noted above, after sequencing macaque immunoglobulin light and heavy variable region genes, it was found that the sequence of each gene family was 85-88% homologous to its human counterpart (Newman et al., 1992).

Essentially, cynomolgus macaque monkeys are administered human CD40L antigen, B cells are isolated therefrom, e.g., lymph node biopsies are taken from the animals, and B lymphocytes are then fused with KH6/B5 (mouse×human) heteromyeloma cells using polyethylene glycol (PEG). Heterohybridomas secreting antibodies which bind human CD40L antigen are then identified.

Antibodies which bind to CD40L or CD40 in a manner which interrupts or regulates CD40 signaling are desirable because such antibodies potentially may be used to inhibit the interaction of CD40L with CD40, with their counter-receptors. If antibodies can be developed against more than one epitope on CD40L or CD40, and the antibodies are utilized together, their combined activity may potentially provide synergistic effects.

The disclosed invention involves the use of an animal which is primed to produce a particular antibody (e.g., primates, such as organgutan, baboons, macaque, and cynomolgus monkeys). Other animals which may be used to raise antibodies to human CD40L include, but are not limited to, the following: mice, rats, guinea pigs, hamsters, monkeys, pigs, goats and rabbits.

A preferred means of generating human antibodies using SCID mice is disclosed in commonly-owned, co-pending U.S. Patent application Ser. No. 08/488,376.

The human genes encoding CD40, CD40L and CD20 antigens have been cloned, and sequenced, and therefore may readily be manufactured by recombinant methods.

Preferably, the human CD40L, CD40 or CD20 antigens will be administered in soluble form, e.g., by expression of the gene encoding the antigen, which has its transmembrane and cytoplasmic domains removed, thereby leaving only the extracellular portion, i.e., the extracellular superfamily V and C-like domains.

Macaques are immunized with CD40L antigen, preferably a soluble form thereof, under conditions which result in the production of antibodies specific thereto. Preferably, the soluble human CD40L antigen will be administered in combination with an adjuvant, e.g., Complete Freund's Adjuvant (CFA), Alum, Saponin, or other known adjuvants, as well as combinations thereof. In general, this will require repeated immunization, e.g., by repeated injection, over several months. For example, administration of soluble human CD40L antigen is effected in adjuvant, with booster immunizations, over a 3 to 4 month period, with resultant production of serum containing antibodies which bound human CD40L antigen.

After immunization, B cells are collected, e.g., by lymph node biopsies taken from the immunized animals and B lymphocytes fused with KH6/B5 (mouse×human) heteromyeloma cells using polyethylene glycol. Methods for preparation of such heteromyelomas are known and may be found in U.S. Ser. No. 08/379,072 by Newman et al., filed on Jan. 25, 1995 and incorporated by reference herein.

Heterohybridomas which secrete antibodies which bind human human CD40L are then identified. This may be effected by known techniques. For example, this may be determined by ELISA or radioimmunoassay using enzyme or radionucleotide labelled human CD40L antigen.

Cell lines which secrete antibodies having the desired specificity to human CD40L antigen are then subcloned to monoclonality.

Cell lines which express antibodies which specifically bind to human CD40L antigen are then used to clone variable domain sequences for the manufacture of primatized antibodies essentially as described in Newman et al., (1992) and Newman et al., U.S. Ser. No. 379,072, filed Jan. 25, 1995, both of which are incorporated by reference herein. Essentially, this entails extraction of RNA therefrom, conversion to cDNA, and amplification thereof by PCR using Ig specific primers. Suitable primers are described in Newman et al., 1992, and in U.S. Ser. No. 379,072.

The cloned monkey variable genes are then inserted into an expression vector which contains human heavy and light chain constant region genes. Preferably, this is effected using a proprietary expression vector of IDEC, Inc., referred to as NEOSPLA. This vector contains the cytomegalovirus promoter/enhancer, the mouse beta globin major promoter, the SV40 origin of replication, the bovine growth hormone polyadenylation sequence, neomycin phosphotransferase exon 1 and exon 2, human immunoglobulin kappa or lambda constant region, the dihydrofolate reductase gene, the human immunoglobulin gamma 1 or gamma 4 PE constant region and leader sequence. This vector has been found to result in very high level expression of primatized antibodies upon incorporation of monkey variable region genes, transfection in CHO cells, followed by selection in G418 containing medium and methotrexate amplification.

For example, this expression system has been previously disclosed to result in primatized antibodies having high avidity (Kd≦10⁻¹⁰M) against CD4 and other human cell surface receptors. Moreover, the antibodies have been found to exhibit the same affinity, specificity and functional activity as the original monkey antibody. This vector system is substantially disclosed in commonly assigned U.S. Ser. No. 379,072, incorporated by reference herein as well as U.S. Ser. No. 08/149,099, filed on Nov. 3, 1993, also incorporated by reference in its entirety herein. This system provides for high expression levels, i.e., >30 pg/cell/day.

The amount of antibody useful to produce a therapeutic effect can be determined by standard techniques well known to those of ordinary skill in the art. The antibodies will generally be provided by standard technique within a pharmaceutically acceptable buffer, and may be administered by any desired route. Because of the efficacy of the presently claimed antibodies and their tolerance by humans it is possible to administer these antibodies repetitively in order to combat various diseases or disease states within a human.

One skilled in the art would be able, by routine experimentation, to determine what an effective, non-toxic amount of antibody would be for the purpose of inducing immunosuppression. Generally, however, an effective dosage will be in the range of about 0.05 to 100 milligrams per kilogram body weight per day.

The antibodies (or fragments thereof) of this invention should also be useful for treating tumors in a mammal. More specifically, they should be useful for reducing tumor size, inhibiting tumor growth and/or prolonging the survival time of tumor-bearing animals. Accordingly, this invention also relates to a method of treating tumors in a human or other animal by administering to such human or animal an effective, non-toxic amount of an antibody. One skilled in the art would be able, by routine experimentation, to determine what an effective, non-toxic amount of anti-CD40L antibody would be for the purpose of treating carcinogenic tumors. Generally, however, an effective dosage is expected to be in the range of about 0.05 to 100 milligrams per kilogram body weight per day.

The antibodies of the invention may be administered to a human or other animal in accordance with the aforementioned methods of treatment in an amount sufficient to produce such effect to a therapeutic or prophylactic degree. Such antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

The route of administration of the antibody (or fragment thereof) of the invention may be oral, parenteral, by inhalation or topical. The term parenteral as used herein includes intravenous, intraperitoneal, intramuscular, subcutaneous, rectal or vaginal administration. The subcutaneous and intramuscular forms of parenteral administration are generally preferred.

The daily parenteral and oral dosage regimens for employing compounds of the invention to prophylactically or therapeutically induce immunosuppression, or to therapeutically treat carcinogenic tumors will generally be in the range of about 0.05 to 100, but preferably about 0.5 to 10, milligrams per kilogram body weight per day.

The antibodies of the invention may also be administered by inhalation. By “inhalation” is meant intranasal and oral inhalation administration. Appropriate dosage forms for such administration, such as an aerosol formulation or a metered dose inhaler, may be prepared by conventional techniques. The preferred dosage amount of a compound of the invention to be employed is generally within the range of about 10 to 100 milligrams.

The antibodies of the invention may also be administered topically. By topical administration is meant non-systemic administration and includes the application of an antibody (or fragment thereof) compound of the invention externally to the epidermis, to the buccal cavity and instillation of such an antibody into the ear, eye and nose, and where it does not significantly enter the blood stream. By systemic administration is meant oral, intravenous, intraperitoneal and intramuscular administration. The amount of an antibody required for therapeutic or prophylactic effect will, of course, vary with the antibody chosen, the nature and severity of the condition being treated and the animal undergoing treatment, and is ultimately at the discretion of the physician. A suitable topical dose of an antibody of the invention will generally be within the range of about 1 to 100 milligrams per kilogram body weight daily.

C. Soluble Ligands for CD40L

In addition to antibodies which recognize and bind to CD40L and inhibit its interaction with CD40, other CD40L antagonists are contemplated for use in treating B-cell lymphomas and leukemias, either alone or in combination with other therapies (e.g., radiation or chemotherapeutics). Other CD40L antagonists are soluble forms of a CD40L ligand. A monovalent soluble ligand of CD40L, such as soluble CD40, can bind CD40L, thereby inhibiting the interaction of CD40L with the CD40 on expressed B-cells. The term “soluble” indicates that the ligand is not permanently associated with a cell membrane. A soluble CD40L ligand can be prepared by chemical synthesis, or, preferably by recombinant DNA techniques, for example by expressing only the extracellular domain (absent the transmembrane and cytoplasmic domains) of the ligand. A preferred soluble CD40L ligand is soluble CD40. Alternatively, a soluble CD40L ligand can be in the form of a fusion protein. Such a fusion protein comprises at least a portion of the CD40L ligand attached to a second molecule. For example, CD40 can be expressed as a fusion protein with an immunoglobulin (i.e., a CD40 Ig fusion protein). In one embodiment, a fusion protein is produced comprising amino acid residues of an extracellular domain portion of the CD40 molecule joined to amino acid residues of a sequence corresponding to the hinge, C_{H}2 and C_{H}3 regions, of an immunoglobulin heavy chain, e.g., Cα1, to form a CD40Ig fusion protein (see e.g., Linsley et al., J. Exp. Med. 1783: 721-730 (1991); Capon et al., Nature 337: 525-531 (1989); and U.S. Pat. No. 5,116,964 (1992)). Such fusion proteins can be produced by chemical synthesis, or, preferably by recombinant DNA techniques based on the cDNA of CD40 (Stamenkovic et al., EMBO J. 8: 1403-10 (1989)).

D. Administration of anti-CD40L

A CD40L antagonist is administered to subjects in a biologically compatible form suitable for pharmaceutical administration in vivo. By “biologically compatible form suitable for administration in vivo” is meant a form of the antagonist to be administered in which any toxic effects are outweighed by the therapeutic effects of the protein. The term “subject” as used in the specification is intended to include living organisms in which an immune response can be elicited, e.g., mammals. Examples of preferred subjects include humans, dogs, cats, horses, ungulates, cows, pigs, goats, sheep, mice, rats, and transgenic species thereof. A CD40L antagonist can be administered in any pharmacological form, optionally in a pharmaceutically acceptable carrier. Administration of a therapeutically effective amount of the antagonist is defined as an amount effective, at dosages and for periods of time necessary to achieve the desired result (e.g., inhibition of the progression or proliferation of the lymphoma being treated). For example, a therapeutically active amount of an antagonist of CD40L may vary according to factors such as the disease stage (e.g., stage I versus stage IV), age, sex, medical complications (e.g., lymphomas related or arising because of AIDS or other immunosuppressed conditions or diseases) and weight of the subject, and the ability of the antagonist to elicit a desired response in the subject. The dosage regimen may be adjusted to provide the optimum therapeutic response. For example, several divided doses may be administered daily, or the dose may be proportionally reduced as indicated by the exigencies of the therapeutic situation.

The active compound, such as an anti-CD40L antibody, by itself or in combination with other active agents, may be administered in a convenient manner such as by injection (subcutaneous, intramuscularly, intravenous, etc.), oral administration, inhalation, transdermal application or rectal administration. Depending on the route of administration, the active compound may be coated in a material to protect the compound from the action of enzymes, acids and other natural conditions which may inactivate the compound. A preferred route of administration is by intravenous (i.v.) injection.

To administer a CD40L antagonist by other than parenteral administration, it may be necessary to coat the antagonist with, or co-administer the antagonist with, a material to prevent its inactivation. For example, an antagonist can be administered to an individual in an appropriate carrier or diluent, co-administered with enzyme inhibitors or in an appropriate carrier or vector, such as a liposome. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions, as well as conventional liposomes (Strejan et al., J. Neuroimmunol. 7: 27-41 (1984)). Additional pharmaceutically acceptable carriers and excipients are known in the art.

The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which- delays absorption, for example, aluminum monostearate and gelatin.

Sterile injectable solutions can be prepared by incorporating an active compound (e.g., an antagonist of CD40L by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof.

When the active compound is suitably protected, as described above, the protein may be orally administered, for example, with an inert diluent or an assimilable edible carrier. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. All compositions discussed above for use with CD40L antagonists may also comprise supplementary active compounds (e.g., chemotherapeutic agents in anti-CD20 antibodies) in the composition.

E. Administration of anti-CD40L with other agents

Hodgkin's Disease. Approximately 7,500 new cases of Hodgkin's Disease (HD) are diagnosed annually in the United States. Radiation therapy alone has been used to treat stage I, II and even stage III HD. Radiation has also been used in combination with chemotherapy (e.g., ABVD and MOPP). See V. T. DeVita et al., “Hodgkin's Disease,” IN CANCER:PRINCIPLES & PRACTICE OF ONCOLOGY vol. 2, 2142-2283 (DeVita et al., eds., 5th ed. 1997) and the references therein for administration of radiation and chemotherapy protocols for treatment of HD.

Chemotherapy drugs useful for treating HD include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating HD. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13th ed. 1994) and V. T. DeVita et al., (1997) and the references cited therein for standard dosing and scheduling. These therapies can be used unchanged, or altered as needed for a particular patient, in combination with CD40L antagonists by itself or in further combination with anti-CD20 antibodies or fragments thereof.

For relapsed, or resistant HD, conventional-dose salvage combination regimens can be utilized in combination with anti-CD40L antibodies, alone or in conjunction with anti-CD20 antibodies. Examples of conventional-dose salvage combination HD regimens include VABCD (vinblastine, doxorubicin, dacarbazine, lomustine and bleomycine), ABDIC (doxorubicin, bleomycin, dacarbazine, lomustine, prednisone), CBVD (lomustine, bleomycin, vinblastine and dexamethasone), PCVP (vinblastine, procarbazine, cyclophosphamide and prednisone), CEP (lomustine, etoposide and prednimustine), EVA (etoposide, vinblastine and doxorubicin), MOPLACE (cyclophosphamide, etoposide, prednisone, methotrexate, cytarabine and vincristine), MIME (methyl GAG, ifosfamide, methotrexate and etoposide), MINE (mitoquazone, ifosfamide, vinorelbine and etoposide), MTX-CHOP (methotrexate and CHOP), CEM (lomustine, etoposide and methotrexate), CAVP (lomustine, melphalan, etoposide and prednisone), EVAP (etoposide, vinblastine, cytarabine and cisplatin), EPOCH (etoposide, vincristine, doxorubicin, cyclophosphamide and prednisone) using the dosages and scheduling as described in V. T. DeVita et al., (1997).

Non-Hodgkin's Lymphoma (NHL). About 40,000 new cases of NHL are diagnosed annually in the U.S., and this number appears to be increasing. Moreover, NHL ranks fourth in the total number of person-years of life lost annually from cancer. NHL comprises several subtypes of lymphomas, with unique clinical presentation and natural history. The breakdown of the NHL subtypes is set forth by a common classification for NHLs, the Working Formulation. Table 1 sets forth the three grades of the Working Formulation.

TABLE 1

| Grade | NHL Subtye |
|---|---|
| Low Grade | Small lymphocytic |
| | Follicular, predominantly small cleaved cell |
| | Follicular, mixed small cleaved and large |
| | cell |
| Intermediate Grade | Follicular, predominantly large cell |
| | Diffuse small cleaved cell |
| | Diffuse mixed small and large cell |
| | Diffuse large cell |
| High Grade | Large cell immunoblastic |
| | Lymphoblastic |
| | Small non-cleaved, Burkitt's and Non- |
| | Burkitt's |
| Other Types | AIDS-related lymphomas |
| | Cutaneous T cell lymphomas |
| | Adult T cell leukemia/lymphoma |
| | Angioimmunoblastic lymphadenopathy |
| | Monocytoid B-cell lymphoma |

The B-cell types of NHL include: small lymphocytic lymphoma/B-cell chronic lymphocytic leukemia (SLL/B-CLL), lymphoplasmacytoid lymphoma (LPL), mantle cell lymphoma (MCL), follicular lymphoma (FL), diffuse large cell lymphoma (DLCL) and Burkitt's lymphoma (BL). See Gaidano et al., “Lymphomas,” IN CANCER: PRINCIPLES & PRACTICE OF ONCOLOGY vol. 2, 2131-2145 (DeVita et al., eds., 5th ed. 1997). Two other formulations (e.g., Kiel formulation and the Revised European American Lymphoma Classification, or REAL) are also used in oncology, and the names of the NHLs may vary as between the two classification systems. See M.A. Shipp et al., “Non-Hodgkin's Lymphomas,” IN CANCER: PRINCIPLES & PRACTICE OF ONCOLOGY vol. 2, 2165-2220 (DeVita et al., eds., 5th ed. 1997). The NHL lymphomas can be further classified by age of patient in which it is diagnosed:

TABLE 2

| | CHILDHOOD B-CELL |
|---|---|
| ADULT B-CELL LYMPHOMAS | LYMPHOMAS |
| Follicular lymphoma | Burkitt's lymphoma |
| Diffuse large B-cell lymphoma | Diffuse large B-cell lymphoma |
| Mantle cell lymphoma | Follicular lymphoma |
| B-CLL/SLL | Precursor B-LBL |
| Immunocytoma/Waldenstrom's | |
| MALT-type/monocytoid B-cell | |

See M.A. Shipp et al., (1997).

Radiotherapy is typically limited to treating patients diagnosed with stage I or II low-grade NHL, and as a potential curative modality for patients who have been aggressively staged. This invention contemplates combining a CD40L antagonist with radiotherapy, and also other cancer treatment modalities to treat NHL.

Chemotherapy is used for most patients with stage II and all patients with stages III and IV disease. Regimens include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). See Shipp et al. (1997), for standard dosages and scheduling. CHOP has also been combined with bleomycin, methotrexate, procarbazine, nitrogen mustard, cytosine arabinoside and etoposide. Less commonly used drugs for treating NHL include: 2-chlorodeoxyadenosine (2-CDA), 2′-deoxycoformycin and fludarabine. For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. Arnold S. Freedman and Lee M. Nadler, Malignant Lymphomas, in HARRISON'S PRINCIPLES OF INTERNAL MEDICINE 1774-1788. In the instance of relapsed, aggressive forms of NHL, the following protocols are recommended IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) using the dosing and schedules described in Shipp et al., (1997).

Recommended protocols by histology and stage for pediatric NHL is as follows:

TABLE 3

| Histology | Protocol |
|---|---|
| LYMPHOBLASTIC | |
| Stage 1 | CHOP |
| Stage 2 | COMP |
| Stage 3 | APO |
| Stage 4 | LSA₂L₂, NHL-BFM 86 |
| SMALL NON-CLEAVED | |
| CELL OR | |
| BURKITT'S LYMPHOMA | |
| Stage 1 | CHOP |
| Stage 2 | COMP |
| Stage 3 | NHL-BFM 86, St. Jude Total B, LMB 89 |
| Stage 4/B-ALL | LMB 89 |
| LARGE CELL | |
| Stage 1 | CHOP |
| Stage 2 | COMP |
| Stage 3 | APO |
| Stage 4 | NHL-BFM 86, ACOP |

APO=doxorubicin, prednisone and vincristine; COMP=cyclophosphamide, oncovin, methotrexate and prednisone. See H. J. Weinstein et al., “Leukemias and Lymphomas of Childhood,” IN CANCER: PRINCIPLES & PRACTICE OF ONCOLOGY vol. 2, 2145-2165 (DeVita et al., eds., 5th ed. 1997) and the references cited therein, all of which are herein incorporated by reference.

Anti-CD40L antibodies and antagonists can be used in combination with any of the chemotherapeutics and/or radiotherapies currently in use for the treatment of HD or the NHL subtype diagnosed. Anti-CD20 antibodies can also be added to the cocktail of therapeutics used. The amount of chemotherapeutic to be used in combination with anti-CD40L antibodies or CD40L antagonists may vary by subject or may be administered according to what is known in the art. See for example, Bruce A Chabner et al., Antineoplastic Agents, in GOODMAN & GILMAN's THE PHARMACOLOGICAL BASIS OF THERAPEUTICS 1233-1287 ((Joel G. Hardman et al., eds., 9th ed. 1996).

Leukemias and other malignancies. The contemplated therapies and methods of treating of the instant invention can also be used to treat B-cell leukemias, including ALL-L3 (Burkitt's type leukemia) and chronic lymphocytic leukemia (CLL), as well as monocytic cell leukemias and other malignancies which express CD40.

Treatment for ALL includes use of vincristine and prednisone. Anthracycline, cyclophosphamide, L-asparaginase have also been added to these treatments. Other induction therapies include four-drug (vincristine, prednisone, anthracycline and cyclophosphamide or asparginase) or five-drug (vincristine, prednisone, anthracycline, cyclophosphamide and asparginase) combinations. For additional therapies and dosages, see D. A. Scheinberg et al., “Acute Leukemias,” IN CANCER: PRINCIPLES & PRACTICE OF ONCOLOGY vol. 2, 2193-2321 (DeVita et al., eds., 5th ed. 1997).

Treatment for CLL includes CMP, CVP, CHOP, COP, and CAP (cyclophosphamide, doxorubicin and prednisone) chemotherapeutic combinations. Treatment in patients with refractory CLL includes the use of purine analogs (e.g., fludarabine monophosphate, 2-chlorodeoxyadenosine and pentostatin). See A.B. Deisseroth et al., “Chronic Leukemias,” IN CANCER: PRINCIPLES & PRACTICE OF ONCOLOGY vol. 2, 2193-2321 (DeVita et al., eds., 5th ed. 1997).

Anti-CD20 and anti-CD40 Antibodies. This invention further contemplates combining anti-CD40L antibodies, such as IDEC-131, with anti-CD20 antibodies or therapeutically effective fragments thereof and/or anti-CD40 antibodies or therapeutically effective fragments thereof. Preferred anti-CD20 antibodies are RITUXAN® and B1 (see U.S. Pat. No. 5,843,398). For description, preparation and using anti-CD40L (also known as anti-gp39), see commonly assigned U.S. patent application Ser. Nos. 08/554,840 filed Nov. 7, 1995, 08/925,339 filed Sep. 8, 1997, 09/069,871 filed Apr. 30, 1998 and 09/332,595 filed Jun. 14, 1999 and U.S. Pat. No. 5,747,037. Preferred anti-CD40 antibodies, and their preparation are described in U.S. Pat. Nos. 5,874,085; 5,874,082; 5,801,227; 5,667,165; 5,674,492 and 5,667,165 all of which are herein incorporated by reference in their entirety. All discussion to anti-CD20 antibodies as described herein, apply as well to anti-CD40 antibodies.

Because peripheral blood B-cell disorders, by definition, can indicate a necessity for access to the blood for treatment, the route of administration of the immunologically active chimeric anti-CD20 antibodies and radiolabeled anti-CD20 antibodies is preferably parenteral; as used herein, the term “parenteral” includes intravenous, intramuscular, subcutaneous, rectal, vaginal or intraperitoneal administration. Of these, intravenous administration is most preferred.

Immunologically active chimeric anti-CD20 antibodies and radiolabeled anti-CD20 antibodies will typically be provided using standard techniques within a pharmaceutically acceptable buffer, for example, sterile saline, sterile buffered water, propylene glycol, combinations of the foregoing, etc. Methods for preparing parenterally administrable agents are described in PHARMACEUTICAL CARRIERS & FORMULATIONS, Martin, Remington's Pharmaceutical Sciences, 15th Ed. (Mack Pub. Co., Easton, Pa. 1975), which is incorporated herein by reference, or as described above.

The specific, therapeutically effective amount of immunologically active chimeric anti-CD20 antibodies useful to produce a unique therapeutic effect in any given patient can be determined by standard techniques well known to those of ordinary skill in the art. Effective dosages (i.e., therapeutically effective amounts) of the immunologically active chimeric anti-CD20 antibodies range from about 0.001 to about 30 mg/kg body weight, more preferably from about 0.01 to about 25 mg/kg body weight, and most preferably from about 0.4 to about 20.0 mg/kg body weight. Other dosages are also viable. Factors influencing dosage include, but are not limited to, the severity of the disease; previous treatment approaches; overall health of the patient; age of the patient; other diseases present, etc. The skilled artisan is readily credited with assessing a particular patient and determining a suitable dosage that falls within the ranges, or if necessary, outside of the ranges as needed. Introduction of the immunologically active chimeric anti-CD20 antibodies or other CD20 antibody (e.g., RITUXAN® or B1) in these dose ranges can be carried out as a single treatment or over a series of treatments. With respect to chimeric antibodies, it is preferred that such administration be carried out over a series of treatments. This preferred approach is predicated upon the treatment methodology associated with this disease. In effect, while a single dosage provides benefits and can be effectively utilized for disease treatment/management, a preferred treatment course can occur over several stages; most preferably, between about 0.4 and between about 20 mg/kg body weight of the immunologically active chimeric anti-CD20 antibody or another anti-CD20 antibody is introduced to the patient once a week for between about 2 to about 10 weeks, most preferably for about 4 weeks.

With reference to the use of radiolabeled anti-CD20 antibodies and/or anti-CD40 antibodes, a preference is that the antibody is (1) non-chimeric or (2) domain-deleted, chimeric humanized antibodies or (3) human antibodies. This preference is predicted upon the significantly shorter circulating half-life of domain-deleted or murine antibodies as compared with whole chimeric or humanized antibodies (i.e., with a longer circulating half-life, the radionuclide is present in the patient for extended periods). However, radiolabeled chimeric antibodies can be beneficially utilized with lower milli-curies (“mCi”) dosages used in conjunction with the unlabeled chimeric antibody relative to the antibody. This scenario allows for a decrease in bone marrow toxicity to an acceptable level, while maintaining therapeutic utility. Specific radiolabeled chimeric forms of anti-CD20 can be prepared as disclosed in U.S. Pat. Nos. 5,776,456 and 5,843,439.

Effective single treatment dosages (i.e., therapeutically effective amounts) of yttrium-90 labeled anti-CD20 antibodies range from between about 5 to about 120 mCi, more preferably between about 10 to about 40 mCi for murine antibodies and about 30 to about 100 mCi for domain deleted antibodies. Effective single treatment non-marrow ablative dosages of iodine-131 labeled anti-CD20 antibodies range from between about 5 to about 70 mCi, more preferably between about 5 to about 40 mCi. Effective single treatment ablative dosages (i.e., may require autologous bone marrow transplantation) of iodine-131 labeled anti-CD20 antibodies range from between about 30 to about 600 mCi, more preferably between about 50 to less than about 500 mCi. In conjunction with a chimeric anti-CD20 antibody, owing to the longer circulating half life compared to murine antibodies, an effective single treatment non-marrow ablative dosages of iodine-131 labeled chimeric anti-CD20 antibodies range from between about 5 to about 40 mCi, more preferably less than about 30 mCi. Imaging criteria for, e.g., the indium- 111 label, are typically less than about 5 mCi. Additional discussion on making and using radiolabeled anti-CD20 antibodies can be found in U.S. Pat. Nos. 5,843,398 and 5,843,439, both of which are hereby incorporated by reference in their entirety.

Radiolabeled Antibodies. With reference to the use of radiolabeled antibodies (e.g., specific to CD40, CD40L and/or CD20), a preference is that the antibody is non-chimeric; this preference is predicted upon the significantly longer circulating half-life of chimeric antibodies vis-a-vis murine antibodies (i.e., with a longer circulating half-life, the radionuclide is present in the patient for extended periods). However, radiolabeled chimeric antibodies can be beneficially utilized with lower milli-Curries (“mCi”) dosages used in conjunction with the chimeric antibody relative to the murine antibody. This scenario allows for a decrease in bone marrow toxicity to an acceptable level, while maintaining therapeutic utility.

A variety of radionuclides are applicable to the present invention and those skilled in the art are credited with the ability to readily determine which radionuclide is most appropriate under a variety of circumstances. For example, iodine-131 (¹³¹I) is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as indium-131 (¹³¹In) and yttrium-90 (⁹⁰Y). ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target antigen.

One non-therapeutic limitation to ⁹⁰Y is based upon the absence of significant gamma radiation making imaging therewith difficult. To avoid this problem, a diagnostic “imaging” radionuclide, such as indium-111(¹¹¹In), can be utilized for determining the location and relative size of a tumor prior to the administration of therapeutic does of ⁹⁰Y-labeled anti-CD20. Indium-111 is particularly preferred as the diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

Effective single treatment dosages (i.e., therapeutically effective amounts) of ⁹⁰Y-labeled antibodies (e.g., anti-CD40L, anti-CD20 and anti-CD40 antibodies) range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-a-vis murine antibodies, an effective single treatment non-marrow ablative dosages of iodine-131 (¹³¹I) labeled chimeric antibodies range from between about 5 and about 40 mCi, more preferably less than about 30 mCi. Imaging criteria for, e.g., the ¹¹¹In label, are typically less than about 5 mCi.

With respect to radiolabeled antibodies for therapy, dosages can also occur using a single therapy treatment or using multiple treatments. Because of the radionuclide component, it is preferred that prior to treatment, peripheral stem cells (“PSC”) or bone marrow (“BM”) be “harvested” for patients experiencing potentially fatal bone marrow toxicity resulting from radiation. BM and/or PSC are harvested using standard techniques, and then purged and frozen for possible reinfusion. Additionally, it is most preferred that prior to treatment a diagnostic dosimetry study using a diagnostic labeled antibody (e.g., using ¹¹¹In) be conducted on the patient, a purpose of which is to ensure that the therapeutically labeled antibody (e.g., using ⁹⁰Y) will not become unnecessarily “concentrated” in any normal organ or tissue.

Additional radioisotopes which may be utilized include ¹²³I, ¹²⁵I, ¹³¹In, ³²P, ⁶⁴Cu, ⁶⁷Cu, ^{211 At,} ¹⁷⁷Lu, ⁹⁰Y, ¹⁸⁶Re, ²¹²Pb, ²¹²Bi, ⁴⁷Sc, ¹⁰⁵Pd, ¹⁰⁹ Pd, ¹⁵³Sm, ¹⁸⁸Re, ¹⁹⁹ Au, ²¹¹At, and ²¹³Bi. The amount of radiation delivered will depend, in part, on half-life and the type, particle emission.

The following materials and methods were used in the experiments described below. The examples provided below do not limit the invention as described or claimed, but merely supply of embodiments of the claimed invention.

### EXAMPLES

### Example 1

### Properties of B lymphoma cells, DHL-4 cells

The concept that anti-CD40L antibody could block CD40L-CD40 mediated survival of malignant B-cells from chemotherapy induced toxicity/apoptosis was tested in vitro using IDEC-131, and the B-lymphoma cell line, DHL-4 (Roos et al., Leuk. Res. 10: 195-202 (1986)) exposed to adriamycin (ADM). IDEC-131 is a humanized version of the murine, monoclonal anti-human CD40L antibody, 24-31.

Initially, the minimum concentration of ADM cytotoxic to DHL-4 cells was determined by exposing DHL-4 cells for 4 hours to different concentrations of ADM. The cell cytotoxicity of DHL-4 cells after 5 days in culture was measured by Alamar Blue, a dye-reduction assay by live cells (see Gazzano-Santoro et al., J. Immunol. Meth. 202: 163-171 (1997)). Briefly, 1×10⁵ DHL-4 cells in growth medium (RMPI-1640 plus 10% Fetal Calf Serum) were incubated with varying concentrations of ADM (1×10⁻⁶ M to 1×10⁻⁸ M) in cell culture tubes at 37° C. for 4 hours. After incubation, cells were washed, re-suspended in growth medium at 1×10⁵ cells/ml concentration and 200 μl of cell suspension was added to each well of 96-well flat-bottom plate. Plates were incubated at 37° C. and tested for cytotoxicity at different time points. During the last 18 hours of incubation, 50 μl of redox dye Alamar Blue (Biosource International, Cat. #DAL 1100) was added to each well. Following incubation, plates were cooled by incubating at room temperature for 10 minutes on a shaker, and the intracellular reduction of the dye was determined. Fluorescence was read using a 96-well fluorometer with excitation at 530 nm and emission at 590 nm. The results are expressed as relative fluorescence units (RFU). The percentage of cytotoxicity was calculated as follows:

[1−(average RFU of test sample÷ Average RFU of control cells)]×100%. Titration curve of ADM cytotoxicity was established and minimal concentrations of the drug for cytotoxicity was selected for subsequent assays.

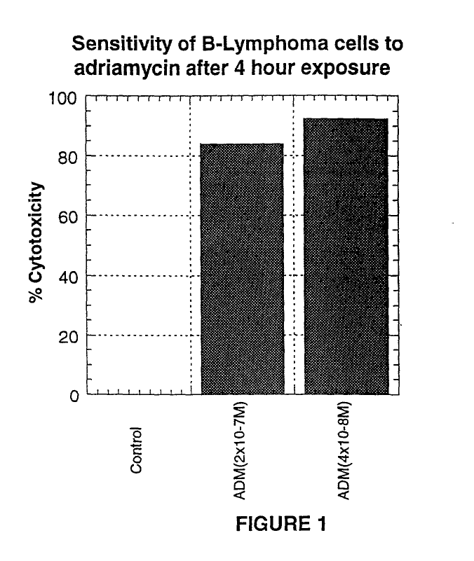

The results, as displayed in FIG. 1, shows cell cytotoxicity of DHL-4 cells cultured for 5 days after being exposed to ADM (2×10⁻⁷ M and 4×10¹⁰⁻⁸ M of ADM) for 4 hours prior to culture. Cells were washed once after exposure and cultured in growth medium for 5 days and cytotoxicity determined by Alamar Blue dye-uptake assay, as described above. Additionally, the DHL-4 cells were characterized for the membrane expression of selected CD molecules by flow cytometry. DHL-4 cells have been found to express CDl9, CD20, CD40 molecules, but no expression of CD40L was detected.

### Example 2

### Anti-CD40L antibody overrides CD40L mediated resistance to killing by to killing by adriamycin of B-lymphoma cells

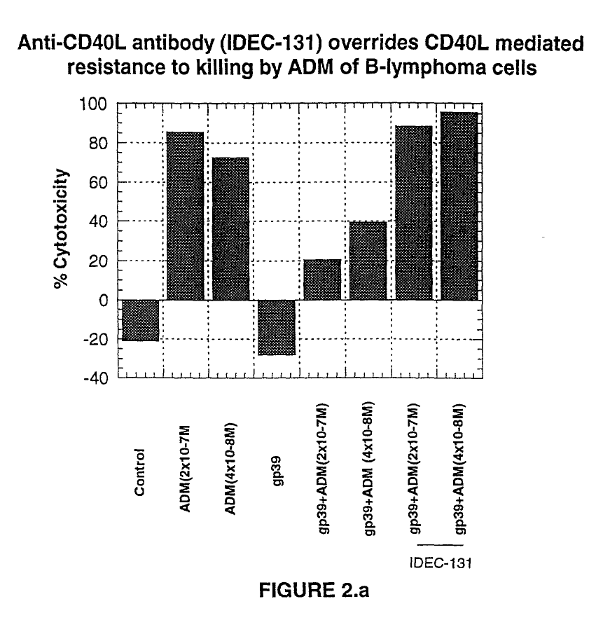

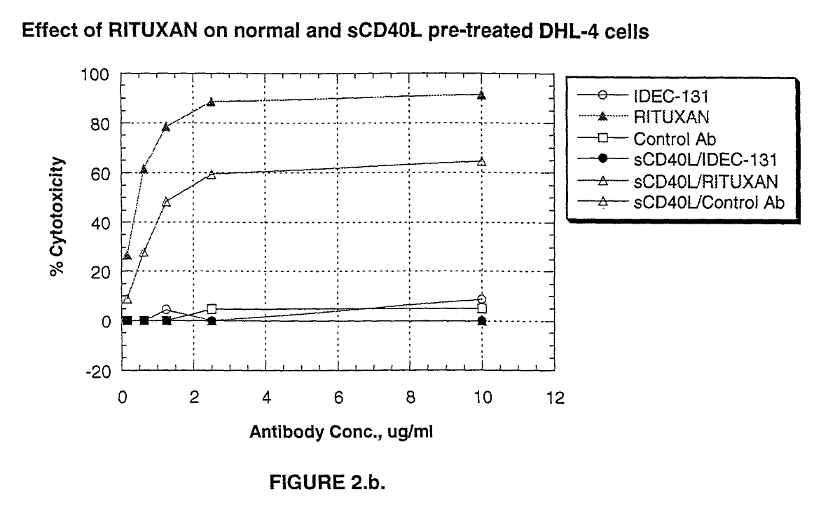

FIG. 2A shows the effect of an anti-CD40L antibody (IDEC- 131) on CD40L-CD40 mediated resistance of DHL-4 cells to cell death induced by ADM. DHL-4 cells (0.5×10⁶ cells/ml) were incubated in the presence of 10 μg/ml of soluble CD40L (sCD40L, P. A. Brams, E. A. Padlan, K. Hariharan, K. Slater, J. Leonard, R. Noelle, and R. Newman, “A humanized anti-human CD 154 monoclonal antibody blocks CD 154-CD40 mediated human B cell activation,” (manuscript submitted)) for 1 hour at 37° C. After 1 hour of incubation, low concentrations of ADM (2×10⁻⁷ M−4×10⁻⁸ M) were added and incubated for another 4 hours in the presence or absence of CD40L (10 μg/ml). Following exposure to ADM, cells were washed and resuspended in growth medium at 0.×10⁶ cells/ml concentration, and 100 μl of cell suspension added to each well of 96-well flat bottom plate, in duplicate, with or without sCD40L. sCD40L (10 μg/ml) was added to cultures that have been continuously exposed to sCD40L during ADM treatment and to cultures that had no sCD40L during ADM exposure. In addition, IDEC-131 at 10 μg/ml was added to cultures to determine its effect on DHL-4 cells incubated with sCD40L and ADM. After 5 days, the cytotoxicity was measured by Alamar Blue dye-uptake assay, as described.

Data show that sCD40L prolonged survival of DHL-4 cells after ADM treatment, whereas, as expected, increased cytotoxicity was observed in cells that were exposed to ADM in the absence of sCD40L. Furthermore, addition of anti-CD40L antibody (IDEC-131) reversed CD40L mediated cell survival, leading to increase in cell cytotoxicity (FIG. 2A).

The addition of IDEC-131 alone had no effect on DHL-4 cells treated with sCD40L, which indicates that the antibody, by itself, does not have any direct inhibitory or cytotoxic activities on DHL-4 cells (FIG. 2B). DHL-4 cells pre-incubated with and without sCD40L were cultured in the presence of different concentrations of IDEC-131, RITUXAN®, the anti-CD20 antibody CE9. 1, and anti-CD4 antibodies (Anderson et al., Clin. Immunol. & Immunopathol. 84: 73-84 (1997)). After 5 days, the cytotoxicity/proliferation of DHL-4 cells was determined by Alamar Blue assay, as described above. FIG. 2B shows no effect on the proliferation or the cytotoxicity of DHL-4 cells by IDEC-13 1, whereas RITUXAN®, as expected, inhibited cell proliferation and induced cytotoxicity. No effect was seen in the DHL-4 cells cultured with anti-CD4 antibodies.

### Example 3

### CD40L-CD40 signaling prevents apoptosis of B-lymphoma cells by anti-CD20 antibody, RITUXAN®

The effect of CD40L-CD40 mediated signaling on anti-CD20 antibody induced apoptosis of B-lymphoma cells was determined using an in vitro system involving DHL-4 cells and the surface cross-linking of RITUXAN®. DHL-4 cells (0.5 to 1×10⁶ cells/ml) were cultured with sCD40L (10 μg/ml) at 37° C. After overnight culture, cells were harvested and incubated with 10 μg/ml of RITUXAN® or the control antibody (CE9.1; an anti-CD4 antibody) with or without sCD40L (10 μg/ml) on ice. After 1 hour of incubation, cells were centrifuged to remove unbound antibodies, and resuspended at 1×10⁶ cells/ml in growth medium (5% FCS-RPMI) and cultured in tissue culture tubes. The cells surface bound antibodies were cross-linked by spiking F(ab′)₂ fragments of goat anti-human Ig-Fcγ specific antibodies at 15 μg/ml, and the cultures were incubated at 37° C. until assayed for apoptosis. Apoptosis was detected using a flow cytometry caspase-3 assay. Cultured cells were harvested at 4 and 24 hours, washed and fixed at 4° C. using Cytofix (Cytofix/Cytoperm™ Kit, Pharmingen Cat. #2075KK). After 20 min of fixation, cells were washed and 15 μl of affinity purified PE-conjugated polyclonal rabbit anti-caspase-3 antibody (Pharmingen, Cat. # 67345) and 50 μl of cytoperm (Pharmingen; Cat. #2075KK) were added. Cells were incubated on ice in the dark for 30 min. After incubation cells were washed once and resuspended in cytoperm. Flow cytometry data was acquired on FACScan and analyzed using WinList software from Verity Software House.

Table I shows resistance of RITUXAN® induced apoptosis in DHL-4 lymphoma cells by exposure to sCD40L. In these studies, activation of caspase-3 was used as the surrogate marker since our previous studies revealed good correlation between caspase-3 and Tunel assay. Cross-linking of RITUXAN® on the DHL-4 cell surface in the presence of sCD40L decreased levels of apoptosis, whereas cells not exposed to sCD40L apoptosed. In comparison, cultures incubated in the presence of an antibody of the same isotype, control antibody (CE9.1), resulted in no apoptosis of the cells. Thus, the data suggests that sCD40L induced signaling of CD40 pathway can lead to development of RITUXAN® mediated killing of B-lymphoma cells.

TABLE I
Resistance of RITUXAN ® mediated
apoptosis of DHL-4 cells by sCD40L

| | % Apoptosis (MIF)^{(a)} | |
|---|---|---|
| Culture Conditions | 4 Hours | 24 Hours |
| DHL-4 cells exposed to sCD40L | | |
| Cells only | 3.35 (17.42) | 4.94 (7.62) |
| Cells + RITUXAN | 1.97 (1.97) | 4.54 (6.54) |
| Cells + RITUXAN + | 21.17 (17.39) | 9.62 (13.44) |
| anti-hu.IgG.F(ab′)₂ | | |
| Cells + CE9.1 | 2.31 (13.25) | 4.15 (7.85) |
| Cells + CE9.1 + anti-hu.IgG.F(ab′)₂ | 2.09 (22.14) | 4.14 (9.57) |
| Cells + anti-hu.IgG.F(ab′)₂ | 1.93 (12.57) | 5.13 (8.02) |
| DHL-4 cells not exposed to sCD40L | | |
| Cells only | 4.36 (14.34) | 5.08 (17.62) |
| Cells + RITUXAN | 5.67 (10.66) | 1.08 (17.92) |
| Cells + RITUXAN + | 74.82 (22.80) | 30.63 (26.84) |
| anti-hu.IgG.F(ab′)₂ | | |
| Cells + CE9.1 | 5.99 (14.00) | 3.05 (18.24) |
| Cells + CE9.1 + anti-hu.IgG.F(ab′)₂ | 5.96 (12.11) | 2.24 (18.19) |
| Cells + anti-hu.IgG.F(ab′)₂ | 6.09 (12.27) | 1.85 (17.27) |

^{(a)}Percent positive cells with caspase-3 activity and its mean fluorescent intensity in log scale.

### Example 4

### Effect of IDEC- 131 on the survival of chronic lymphocytic leukemia (CLL) cells

To determine the effect of IDEC- 131 on the growth and survival of B-CLL cells in vitro, B-CLL cells were cultured with and without IDEC- 131 in the presence of CD40L in vitro. Peripheral blood mononuclear cells (PBMC) were isolated from a CLL patient's blood using a Ficoll-Hypaque gradient centrifugation. Viability was determined by Trypan blue dye exclusion and was >98%. Flow cytometric analysis revealed that >70% of the lymphocytes were CD19+/CD20+. CLL cells (PBMC) were cultured in CLL growth medium (e.g., RPMI-1640 medium supplemented with 5% FCS or 2% of autologous donor plasma, supplemented with 2 mM L-Glutamine and 100 U/ml Penicillin-Streptomycin). In addition, for some experiments, CD 19+ B-cells were purified using CD19+ Dynabeads™ as per manufacture's instructions (Dynal, Cat. #111.03/111.04) and cultured as above. CLL or purified B-CLL cells cultured in growth medium mostly under went spontaneous apoptotic cell death. However, culturing these cells in the presence of sCD40L extended their viability in cultures. Table II indicates the cell viability of CD19+ B-CLL cells grown in the presence or absence of sCD40L (5 μg/ml) at different time points and indicates the longer survival of CLL cells. B-CLL cells from Patient #1 cultured with sCD40L had ≧60% viability for greater than 2 weeks, whereas cells grown in the absence of sCD40L had less than 10% viability.

TABLE II
Survival of B-CLL cells in the presence of sCD40L

| | | |
|---|---|---|
| B-CLL | Time | % Viability^{(a)} |

| Sample | (Hours) | (−) CD40L | (+) CD40L |
|---|---|---|---|
| Patient #1 | 0 | ≧90 | ≧90 |
| | 48 | 88 | 90 |
| | 96 | 46 | 77 |
| | 144 | 30 | 72 |
| Patient #2 | 0 | ≧90 | ≧90 |
| | 72 | 40 | 72 |
| | 96 | 31 | 65 |
| | 144 | 17 | 51 |

^{(a)}equals the percent viability determined by Trypan blue dye exclusion.

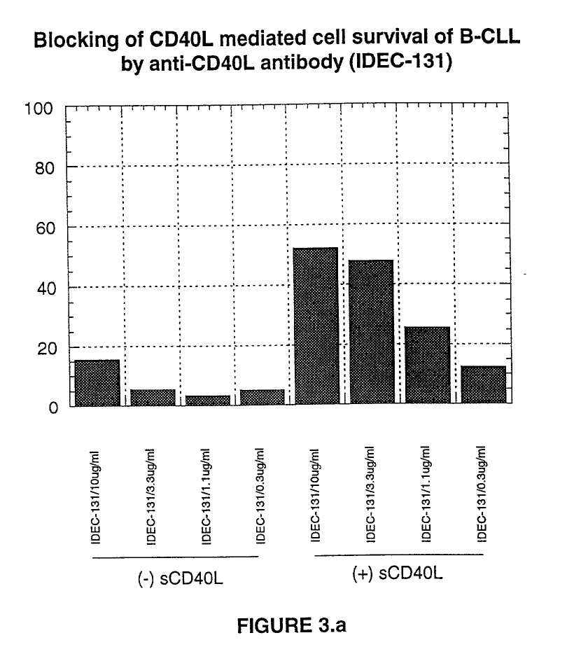

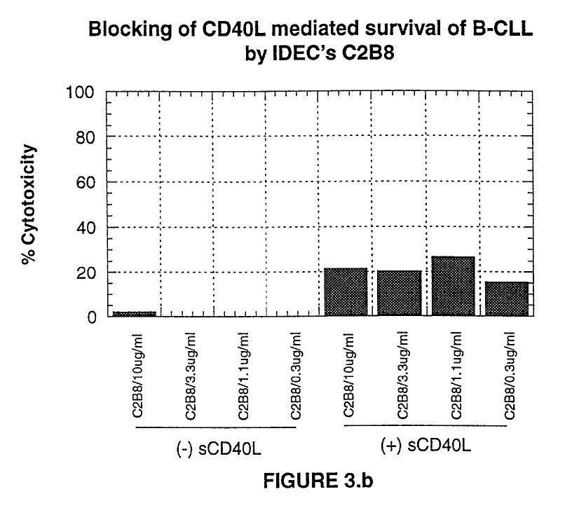

FIG. 3A shows the effect of IDEC-131 on the growth and survival of B-CLL cells after 7 days in culture. Purified B-CLL cells from a CLL patient (2×10⁶ cells/ml) were divided into two culture tubes. Cells in one tube were mixed with sCD40L (5 μg/ml) in equal volume of growth medium, whereas the other tube was incubated with equal volume of growth medium as control. After 1 hour of incubation at 37° C., cells were gently mixed and 100 μl of cell suspension media added to each well of a 96-well flat bottom plate in duplicate with and without varying concentrations of IDEC-131 (10 μg/ml to 0.3 μg/ml). Seven days later, cell survival/death in culture was determined by Alamar Blue assay, as described above. Data showed cell survival in cultures with sCD40L. The addition of IDEC-131 into culture resulted in increased cell death, which indicated a reversal of cell survival or a sensitization to cell death. Additionally, RITUXAN® administered at the same concentration as the IDEC- 131 produced less of lower effect than IDEC-131 on cell death (FIG. 3B).

### Example 5

### CD40L-CD40 mediated up-regulation of HLA-DR molecules in B-CLL

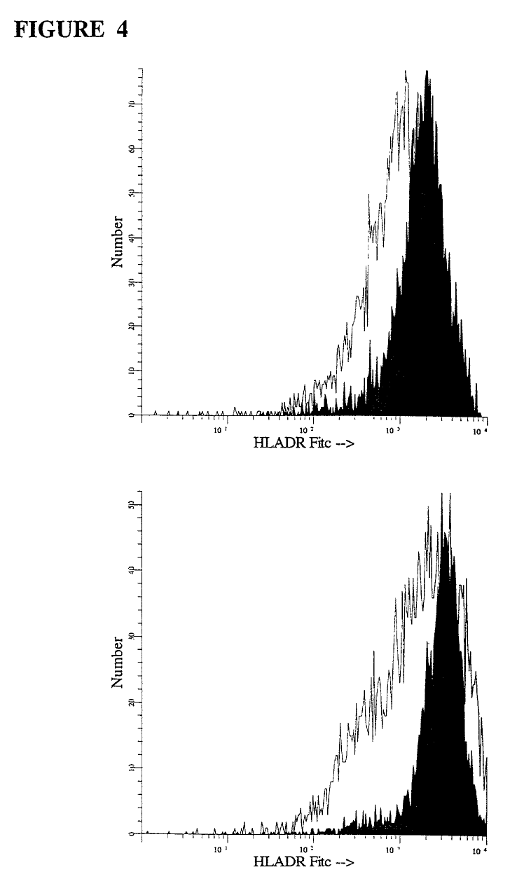

To determine whether the CD40L-CD40 signal transduction pathway is intact, CLL cells from CLL patients were cultured (5×10⁵ cells/ml) with and without 5 μg/ml of CD40L at 37° C. At 48 hours and 144 hours, the class II molecule, HLA-DR expression, was determined on CD 19+ cells by flow cytometry using standard procedures. Briefly, cultured lymphocytes were harvested at different time points and analyzed for surface expression of molecules using antibodies coupled to either fluorescein (FITC) or phycoerythrin (PE) for single or double staining using a FACScan (Becton-Dickinson) flow cytometer. To stain for flow cytometry, 1×10⁶ cells in culture tubes were incubated with appropriate antibodies as follows: anti-CD45-FITC to gate lymphocyte population on a scatter plot; anti-CD19-PE (Pharmingen, Cat. #30655) or anti-CD20-FITC (Pharmingen; Cat. #33264) antibodies to determine the CD19+ and/or CD20+ B-cells; anti-CD3-FITC antibodies (Pharmingen; Cat. #30104) to gate-off the T cells; anti-CD19-RPE and anti-HLA-DR-FITC antibodies (Pharmingen; Cat. #32384) to determine the Pclass II expression on CD 19+ cells. Cells were washed once by centrifugation (at 200×g, for 6 min.) with 2 ml cold PBS and incubated with antibody for 30 min. on ice, after which the cells washed once, fixed in 0.5% paraformaldehyde and stored at 4° C. until analyzed. Flow cytometry data was acquired on FACsan and analyzed using WinList software (Verity Software House). The machine was set to autogating to allow examination of quadrants containing cells that were single stained with either RPE or FITC, unstained or doubly stained. FIG. 4 shows the comparison of HLA-DR expression in CD 19+ CLL cells cultured with sCD40L and those cells not cultured with sCD40L. A higher level of HLA-DR expression was detected on B-CLL cells cultured in the presence of sCD40L (Table III).

TABLE III
CD40L-CD40 mediated up-regulation of HLA-DR molecule in B-CLL

HLA-DR^{+(a)}

| Sample | Time | % Positive | MFI |
|---|---|---|---|
| Control | 48 hrs | 81 | 92 |
| | 144 hrs | 88 | 1655 |
| Cells + sCD40L | 48 hrs | 88 | 101 |
| | 144 hrs | 95 | 2943 |

^{(a)}CD19⁺ B-cells that are positive for HLA-DR molecules and its mean fluorescent intensity (MIF).

### Example 6

### Preparation of IDEC- 131 and RITUXAN®

For treatment of a CD40+ malignancy, IDEC-131 at about 10 to about 50 mg/ml in a formulation buffer 10 mM Na-citrate, 150 mM NaC1, 0.02% Polysorbate 80 at pH 6.5 is infused intravenously (iv) to a subject. IDEC-13 1 is administered before, after or in conjunction with RITUXAN®. The RITUXAN® dosage infused ranges from about 3 to about 10 mg/kg of subject weight.

### Example 7

### Preparation of IDEC- 131 and CHOP

For treatment of CD40+ malignancies responsive to CHOP (e.g., Hodgkin's Disease, Non-Hodgkin's lymphoma and chronic lymphocytic leukemia, as well as salvage therapy for malignancies wherein cells are CD40+), IDEC-131 is infused at a dosage ranging from about 3 to about 10 mg per kg of patient weight immediately prior to the initiation of the CHOP cycle. IDEC-131 administration will be repeated prior to each CHOP cycle for a total of 4 to 8 cycles.

### Example 8

### Administration of anti-CD4L in combination with RITUXAN® to treat B-cell lymphoma in a subject

Combination therapies are particularly useful as salvage therapies or for treating relapsed or aggressive forms of CD40+ malignancies (e.g., Hodgkin's Disease, Non-Hodgkin's lymphoma and CLL). When IDEC- 131 is to be administered in combination with CHOP and RITUXAN®, IDEC-131 is administered as discussed above in Example 6, followed by the schedule specified for CHOP-IDEC-131 administration in Example 7.

All references discussed above are hereby incorporated by reference in their entirety.

What is claimed is:

1. A method for treating CD40+ malignancies comprising administering a therapeutically effective amount of an antibody or antibody fragment which binds to CD40L thereby inhibiting CD40/CD40L interaction or CD40 signaling.

2. The method of claim 1, wherein the CD40+ malignancy is a B-cell lymphoma or a B-cell leukemia.

3. The method of claim 2, wherein the B-cell lymphoma is Hodgkin's Disease (HD) or Non-Hodgkin's Lymphoma (NHL).

4. The method of claim 3, wherein the NHL is low grade, intermediate grade or high grade.

5. The method of claim 3, wherein the NHL is selected from the subtype group consisting of: small lymphocytic, follicular and predominantly small cleaved cell, follicular and mixed small cleaved and large cell type, follicular and predominantly large cell type, diffuse small cleaved cell, diffuse mixed small and large cell, diffuse large cell, large cell immunoblastic, lymphoblastic, small non-cleaved Burkitt's and non-Burkitt's type, AIDS-related lymphomas, angioimmunoblastic lymphadenopathy, mantle cell lymphoma, and monocytoid B-cell lymphoma.

6. The method of claim 2, wherein the B-cell leukemia is a chronic B-cell leukemia, acute lymphoblastic leukemia of a B-cell lineage, or chronic lymphocytic leukemia of a B-cell lineage.

7. The method of claim 2, wherein the antibody or antibody fragment which binds to CD40L is IDEC-131, 3E4, 2H5, 2H8, 4D9-8, 4D9-9, 24-31, 24-43, 89-76or 89-79.

8. The method of claim 7, wherein the antibody or antibody fragment is chimeric, bispecific, human or humanized.

9. The method of claim 2, wherein the antibody fragment is Fab, Fab′, scFv or F(ab′)₂.

10. The method of claim 2, further comprising administering a therapeutically effective amount of a second antibody or fragment thereof, a chemotherapeutic, a combination of chemotherapeutic agents and/or a radiotherapy.

11. The method of claim 10, wherein the radiotherapy is external radiation treatment or a radiolabeled antibody.

12. The method of claim 11, wherein the radiolabeled antibody is radiolabeled IDEC-131, RITUXAN®, or B1 or fragments thereof.

13. The method of claim 12, wherein the radiolabeled antibody is radiolabeled with ¹²³I, ¹²⁵I, ¹³¹I, ¹¹¹In, ¹³¹In ³²P, ⁶⁴Cu, ⁶⁷Cu, ²¹¹At, ¹⁷⁷Lu, ⁹⁰Y, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²¹¹At, and ²¹³Bi.

14. The method of claim 10, wherein the chemotherapeutic agent for treating HD is any one or more of the following: an alkylating agent, a vinca alkaloid, procarbazine, methotrexate or prednisone.

15. The method of claim 10, wherein the chemotherapeutic agent for treating NHL is any one or more of the following: an alkylating agent, cyclophosphamide, chlorambucil, 2-CDA, 2′-deoxycofornycin, fludarabine, cytosine arabinoside, cisplatin, etoposide or ifosfamide.

16. The method of claim 10, wherein the combination of chemotherapeutic agents for treating HD is: MOPP, ABVD, ChlVPP, CABS, MOPP plus ABVD, MOPP plus ABV, BCVPP, VABCD, ABDIC, CBVD, PCVP, CEP, EVA, MOPLACE, MIME, MINE, CEM, MTX-CHOP, EVAP or EPOCH.

17. The method of claim 10, wherein the combination of chemotherapeutic agents for treating NHL is: CVP, CHOP, C-MOPP, CAP-BOP, m-BACOD, ProMACE-MOPP, ProMACE-CytaBOM, MACOP-B, IMVP-16, MIME, DHAP, ESHAP, CEPP(B) or CAMP.

18. The method of claim 10, wherein the chemotherapeutic agent for treating a B-cell leukemia is at least one of the following: anthracycline, cyclophosphamide, L-asparginase and a purine analog.

19. The method of claim 10, wherein the combination of chemotherapeutic agents for treating a B-cell leukemia is: vincristine, prednisone, anthracycline and cyclophosphamide or asparginase; vincris tine, prednisone, anthracycline, cyclophosphamide and asparginase; CHOP; CMP; CVP; COP or CAP.

20. The method of claim 10, wherein the second antibody is an anti-CD20 antibody.

21. The method of claim 21, wherein the anti-CD20 antibody is RITUXAN® or a fragment thereof or B1 or a fragment thereof.

22. A method of treating a CD40+ malignancy comprising the step of administering an anti-CD40L antibody or fragment thereof wherein the anti-CD40L antibody or antibody fragment blocks CD40-CD40L interaction or inhibits CD40 signalling; and administering an anti-CD20 antibody or fragment thereof.

23. The method of claim 22, wherein the CD40+ malignancy is a B-cell lymphoma or a B-cell leukemia.

24. A combination therapy for the treatment of a CD40+ malignancy comprising a CD40L antagonist and at least one of the following (a) a chemotherapeutic agent or a combination of chemotherapeutic agents, (b) a radiotherapy, (c) an anti-CD20 antibody or fragment thereof and (d) anti-CD40 antibody or fragment thereof.

25. The method of claim 24, wherein the radiotherapy is external radiation treatment or a radiolabeled antibody.

26. The method of claim 25, wherein the radiolabeled antibody is radiolabeled with ¹²³I, ¹²⁵I, ¹³¹I, ¹¹¹In, ¹³¹In ³²P, ⁶⁴Cu, ⁶⁷Cu, ²¹¹At, ¹⁷⁷Lu, ⁹⁰Y, ¹⁸⁶Re, ²¹²Pb, ²¹²B,i ⁴⁷Sc, ¹⁰⁵Rh, ¹⁰⁹Pd, ¹⁵³Sm, ¹⁸⁸Re, ¹⁹⁹Au, ²¹¹At, and ²¹³Bi.

27. The combination therapy of claim 24 wherein the CD40+ malignancy is a B-cell leukemia or B-cell lymphoma.

28. The combination therapy of claim 27, wherein the B-cell lymphoma is HD or NHL.

29. The combination therapy of claim 28, wherein the NHL is low grade, intermediate grade or high grade.

30. The combination therapy of claim 28, wherein the NHL is selected from the subtype group consisting of the following: small lymphocytic, follicular and predominantly small cleaved cell, follicular and mixed small cleaved and large cell type, fotlicular and predominantly large cell type, diffuse small cleaved cell, diffuse mixed small and large cell, diffuse large cell, large cell immunoblastic, lymphoblastic, small non-cleaved Burkitt's and non-Burkitt's type, AIDS-related lymphomas, angioimmunoblastic lymphadenopathy, mantle cell lymphoma and monocytoid B-cell lymphoma.

31. The combination therapy of claim 28, wherein the B-cell leukemia is a chronic B-cell leukemia, acute lymphoblastic leukemia of a B-cell lineage, or chronic lymphocytic leukemia of a B-cell lineage.

32. The combination therapy of claim 24, wherein the CD40L antagonist is an anti-CD40L antibody or a fragment thereof.

33. The combination therapy of claim 32, wherein the anti-CD40L antibody is IDEC- 131 or a fragment thereof.

34. The combination therapy of claim 32, wherein the anti-CD40L fragment is Fab, Fab′, scFv or F(ab′)₂.

35. The combination therapy of claim 24, wherein the anti-CD20 antibody is RITUXAN® or a fragment thereof or B1 or a fragment thereof.

36. The combination therapy of claim 28, wherein the chemotherapeutic agent for treating HD is any one or more of the following: an alkylating agent, a vinca alkaloid, procarbazine, methotrexate or prednisone.

37. The combination therapy of claim 28, wherein the chemotherapeutic agent for treating NHL is any one or more of the following: an alkylating agent, cyclophosphamide, chlorambucil, 2-CDA, 2′-deoxycoformycin, fludarabine, cytosine arabinoside, cisplatin, etoposide or ifosfamide.

38. The combination therapy of claim 28, wherein the combination of chemotherapeutic agents for treating HD is: MOPP, ABVD, ChlVPP, CABS, MOPP plus ABVD, MOPP plus ABV, BCVPP, VABCD, ABDIC, CBVD, PCVP, CEP, EVA, MOPLACE, MIME, MINE, CEM, MTX-CHOP, EVAP or EPOCH.

39. The combination therapy of claim 28, wherein the combination of chemotherapeutic agents for treating NHL is: CVP, CHOP, C-MOPP, CAP-BOP, m-BACOD, ProMACE-MOPP, ProMACE-CytaBOM, MACOP-B, IMVP- 16, MIME, DHAP, ESHAP, CEPP(B), or CAMP.

40. The combination therapy of claim 28, wherein the chemotherapeutic agent for treating a B-cell leukemia is: anthracycline, cyclophosphamide, L-asparginase, a purine analog.

41. The combination therapy of claim 28, wherein the combination of chemotherapeutic agents for treating a B-cell leukemia is: vincristine, prednisone, anthracycline and cyclophosphamide or asparginase; vincristine, prednisone, anthracycline, cyclophosphamide and asparginase; CHOP; CMP; CVP; COP or CAP.

42. A composition for the treatment of a CD40+ malignancy comprising an (i) anti-CD40L antibody or antibody fragment thereof and at least one of the following: (ii) a radiolabeled antibody that binds CD40L or CD20, (iii) an anti-CD20 antibody or fragment thereof, or (iv) a chemotherapeutic agent or a chemotherapeutic combination.

43. The composition for the treatment of a CD40+ malignancy of claim 42 wherein the malignancy is a B-cell lymphoma or a B-cell leukemia.

44. The composition of claim 43, wherein the B-cell leukemia is Hodkin's Disease or NHL.

45. The composition of claim 42, wherein the radiolabeled antibody is radiolabeled IDEC- 131, RITUXAN®, or B 1.

46. The composition of claim 46, wherein the radiolabeled antibody is radiolabeled with ¹²³I, ¹²⁵I, ¹³¹I, ¹¹¹In, ¹³¹In, ³²P, ⁶⁴Cu, ⁶⁷Cu, ²¹¹At, ¹⁷⁷Lu, ⁹⁰Y, ¹⁸⁶ Re, ²¹²Pb, ²¹²Bi, ⁴⁷Sc, 105Rh, ¹⁰⁹Pd, ¹⁵³Sm, ¹⁸⁸Re, ¹⁹⁹Au , ²¹¹At, and ²¹³Bi.

47. The composition of claim 44, wherein the NHL is low grade, intermediate grade or high grade.

48. The composition of claim 44, wherein the NHL is selected from the NHL subtype group consisting of the following: small lymphocytic, follicular and predominantly small cleaved cell, follicular and mixed small cleaved and large cell type, follicular and predominantly large cell type, diffuse small cleaved cell, diffuse mixed small and large cell, diffuse large cell, large cell immunoblastic, lymphoblastic, small non-cleaved Burkitt's and non-Burkitt's type, AIDS-related lympliomas, angioimmunoblastic lymphadenopathy, mantle cell lymphoma and monocytoid B-cell lymphoma.

49. The composition of claim 42, wherein the anti-CD40 antibody is IDEC-131 or a fragment thereof.

50. The composition of claim 42, wherein the anti-CD20 antibody is RITUXAN® or a fragment thereof or B1 or a fragment thereof.

51. The composition of claim 43, wherein the chemotherapeutic agent for treating HD is any one or more of the following: an alkylating, agent, a vinca alkaloid, procarbazine, methotrexate or prednisone.

52. The composition of claim 44, wherein the chemotherapeutic agent for treating NHL is any one or more of the following: an alkylaing agent, cyclophosphamide, chlorambucil, 2-CDA, 2′-deoxycoformycin, fludarabine, cytosine arabinoside, cisplatin, etoposide or ifosfamide.

53. The composition of claim 44, wherein the combination of chemotherapeutic agents for treating HD is: MOPP, ABVD, ChlVPP, CABS, MOPP plus ABVD, MOPP plus ABV, BCVPP, VABCD, ABDIC, CBVD, PCVP, CEP, EVA, MOPLACE, MIME, MINE, CEM, MTX-CHOP, EVAP or EPOCH.

54. The composition of claim 44, wherein the combination of chemotherapeutic agents for treating NHL is: CVP, CHOP, C-MOPP, CAP-BOP, m-BACOD, ProMACE-MOPP, ProMACE-CytaBOM, MACOP-B, IMVP-16, MIME, DHAP, ESHAP, CEPP(B), or CAMP.

55. The composition of claim 43, wherein the chemotherapeutic agent for treating a B-cell leukemia is: anthracycline, cyclophosphamide, L-asparginase, a purine analog.

56. The composition of claim 43, wherein the combination of chemotherapeutic agents for treating a B-cell leukemia is: vincristine, prednisone, anthracycline and cyclophosphamide or asparginase; vincristine, prednisone, anthracycline, cyclophosphamide and asparginase; CHOP; CMP; CVP; COP or CAP.
